(19) United States
(12) Patent Application Publication
(10) Pub. No.: US 2013/0090630 A1
(43) Pub. Date: Apr. 11, 2013

(54) Insertion facilitation device for catheters

(71) Applicant: Urovalve, inc., Newark, NJ (US)

(72) Inventor: Thomas W. Winegar, Hawthorne, NJ (US)

(73) Assignee: Urovalve, inc., Newark, NJ (US)

(21) Appl. No.: 13/573,702

(22) Filed: Oct. 3, 2012

Related U.S. Application Data
(63) Continuation of application No. 12/881,978, filed on Sep. 14, 2010, now abandoned.
(60) Provisional application No. 61/242,138, filed on Sep. 14, 2009.

(51) Int. Cl. A61M 25/01

(52) U.S. Cl. CPC A61M 25/0102
USPC 604/524

(57) ABSTRACT

Devices that facilitate insertion of tube-like structures, e.g., catheters and other medical devices, into passages in mammalian bodies, e.g. in the human body. Devices according to various embodiments of the invention provide greater stiffness and/or rigidity to elongated tube-like structures. According to some embodiments, a portion or the entire insertion facilitation device can be removed from the tube-like structure after insertion thereof. According to some embodiments of the invention, an insertion facilitation device comprises a releasable engagement arrangement to releasably engage the stiffener and a tube-like structure to inhibit or prevent relative motion between the stiffener and tube-like structure.

### CROSS REFERENCE TO RELATED APPLICATIONS

This application is a continuation of application Ser. No. 12/881,978 filed Sep. 14, 2010 entitled “Insertion Facilitation Device for Catheters,” and claims priority to U.S. Patent Application Ser. No. 61/242,138 filed Sep. 14, 2009 entitled “Insertion Facilitation Device for Catheters,” the disclosures of both applications being incorporated herein by reference.

### BACKGROUND OF THE INVENTION

The invention disclosed herein relates to devices that facilitate insertion of tube-like structures, e.g., catheters and other medical devices, into passages, e.g., in the human or other mammalian body. The invention has application or potential application to many medical fields, including urology, vascular, thoracic, cardiology, gastroenterology and proctology.

Tube-like structures such as catheters, for example, and other devices are inserted into body passages, e.g. of humans, for a variety of purposes. Some currently used tube-like structures, because of material selection, size, shape, or other consideration, lack sufficient stiffness to support the weight of the structure without deflection if the structure is supported at either, or both, ends. Depending upon the amount of deflection, such tube-like structures may or may not be considered self-supporting. Some frequently used tube-like structures may undergo deflection or buckling when being advanced in a passage in response to resistance. Such deflections can hinder insertion of the tube-like structure into a passage in a mammalian body.

Generally, the stiffer a catheter or other device, the easier it is to insert. However, stiffer catheters and other devices present more risk of possible patient trauma and creation of false passages. For example, extreme rigidity in a urinary catheter, while useful for pushing through a blockage, can also result in the catheter being pushed through the wall of the urethra and/or creating a false passage.

International Application No. PCT/US2007/086157, published under WO 2008/067557, describes an insertion tool for a urinary catheter. The entire disclosure of published International Application WO 2008/067557 is incorporated herein by reference. U.S. application Ser. No. 11/993,356 is a US national stage filing of International Application No. PCT/US2007/086157 and is assigned to the assignee of this application

### SUMMARY OF THE INVENTION

Devices according to various embodiments of the invention provide stiffness and/or rigidity or a greater stiffness and/or rigidity to elongated tube-like structures, e.g., provide the tube-like structures with more stiffness and/or rigidity than without such a device. Such a device is sometimes referred to herein as an “insertion facilitation device” or a “tool.” (For ease of description, the terms “stiff” and “stiffness” encompass “rigid” and “rigidity” unless the context indicates otherwise.) Insertion facilitation devices according to various embodiments of the invention, facilitate insertion of a tube-like structure into a passage. For example, tube-like structures including an insertion facilitation device according to various embodiments of the invention resist buckling and/or angular displacement of the tube-like structure at least during insertion thereof in the passage at least sufficiently to permit insertion of the tube-like structure into a passage.

Insertion facilitation devices according to various embodiments of the invention may have a stiffness that is greater than, the same as, or less than the stiffness of the tube-like structure. However, the stiffness of the tube-like structure is greater with the insertion facilitation device than without it. Insertion facilitation devices according to embodiments of the invention may provide stiffness to a tube-like structure in a range up to substantial rigidity, e.g., up to the tube-like structure being self-supporting with little or no droop or sag along all or a substantial portion of its length. For example, in embodiments of tube-like structures comprising a catheter, stiffness may be provided up to that of stylets currently in use with catheters.

A range of stiffnesses may be provided in insertion facilitation devices according to various embodiments of the invention by selection of size, material, wall thickness, etc. of the device or a part thereof. It would be understood by an ordinarily skilled artisan that many factors determine stiffness, e.g., material, cross sectional size and shape, length, etc. Accordingly, a wide range of stiffnesses can be achieved by varying one or more of such and other factors in an insertion facilitation device according to embodiments of the invention.

As a general matter, a tube-like structure into which an insertion facilitation device according to one or more embodiments of the invention has been installed is capable of travelling through, or more easily travelling through, a natural curve or curves in the passage and/or pushing through, or more easily pushing through, a difficult section or sections of a passage, than the tube-like structure without such an insertion device, and yet remains capable of negotiating the passage. For example, the tube-like structure with the insertion device is flexible and stiff enough to negotiate the passage and/or push through a difficult section or sections.

Stiffeners used in a tube-like structure otherwise unable to sufficiently support itself or prone to deflection or buckling during insertion into a passage as described above, may provide sufficient support to render the tube-like structure and the stiffener to be more self supporting and/or to better resist deflection/buckling during insertion into a passage. At the same time, a tube-like structure with such a stiffener is not so stiff as to be incapable of deflecting to follow a passage in which the tube-like structure and stiffener are being inserted. As discussed herein, the degree of deflection during insertion that is considered to be acceptable or desirable depends upon the particular application. In general, it is often desirable for a tube-like structure to possess sufficient stiffness to penetrate blockages within the passage, but not be so stiff as to cause unnecessary trauma to the passage, puncture the passage, or cause false passages.

The range of stiffness achieved in a paired tube-like structure and stiffener may vary, depending upon the requirements of the application and the particular stiffener selected to facilitate insertion of the tube-like structure. Once the tube-like structure is inserted into and positioned in the passage, the passage primarily or entirely supports the tube-like structure and the stiffness of the combined tube-like structure and stiffener which assisted the insertion may not be needed and could cause discomfort in the recipient. According to some embodiments of the invention, provision may be made for withdrawing the stiffener from the tube-like structure.

Insertion facilitation devices according to some embodiments of the invention provide such stiffness to a tube-like structure that is releasable. According to some embodiments, a portion or all of an insertion facilitation device can be removably assembled into a tube-like structure such that the tube-like structure can be inserted into a passage, and then used with or without the insertion facilitation device or portion thereof. For example, in some applications, a tube-like structure after insertion into a passage may suitably be used in the passage without the stiffness an insertion facilitation device provides. According to some embodiments of the invention, a portion or all of the insertion facilitation device can be removed from the tube-like structure after insertion thereof in a passage such that the tube-like structure can be used in the passage without the device or portion thereof. In various embodiments of the invention, after removal of an insertion facilitation device from a tube-like structure, the tube-like structure returns to its state of stiffness before receiving an insertion facilitation device.

Use of an insertion facilitation device according to some embodiments of the invention enables selection of a smaller, less stiff, softer and/or otherwise more desirable tube-like structure, e.g. a catheter, for use in a passage. For example, a smaller, more flexible and/or softer catheter which would present insertion difficulties if insertable at all without an insertion facilitation device may otherwise be used with an insertion facilitation device according to various embodiments of the invention so as to increase the comfort of a patient, for example where the catheter will remain in place for a period of time.

According to some embodiments of the invention, an insertion facilitation device releasably engages with a tube-like structure and provides stiffness or greater stiffness to the tube-like structure. In some embodiments, the releasable engagement substantially reduces or eliminates the risk of the insertion facilitation device dislodging or separating from the tube-like structure at least during insertion thereof in a passage. (“Engage” and formatives thereof as used herein are meant in a broad sense and encompass sufficient holding or retaining, etc., for the purposes described herein, and encompass various forms of attachment, fixation, securement, bonding, integral formation, etc. “Releasably engage” and formatives thereof as used herein are meant in a broad sense and encompass forms of engagement that can be disengaged as disclosed herein. For example, not every form of engagement is also a releasable engagement.) For example, a prior art urinary catheter known as a Foley catheter includes eyelets in the proximal end portion. (For ease of description, the leading end or portion of a tube-like structure, insertion facilitation device or stiffener (discussed below) relative to a passage will be referred to herein as the proximal end or portion, and the trailing end or portion will be referred herein as the distal end or portion.) Use in a Foley-type catheter of an insertion facilitation device according to various embodiments of the invention in which the proximal portion of the device is engaged with the catheter essentially eliminates the risk that the proximal end portion of the device will become dislodged and exit the catheter through an eyelet, or create and protrude through an unintentional opening, which could otherwise pose a risk of trauma to the patient.

According to some embodiments of the invention, an insertion facilitation device comprises an elongated stiffener that in use extends along the tube-like structure and is releasably engaged with the tube-like structure such that risk of axial and/or radial displacement of the stiffener relative to the tube-like structure is substantially reduced or eliminated, e.g., at least during insertion of the tube-like structure in a passage. According to some embodiments, the stiffener may be used as a pusher for the tube-like structure. In some embodiments, the stiffener comprises an elongated member, e.g., a solid or hollow tube or rod or bar or wire, or an assembly of parts to form an elongated assembly. The insertion facilitation device remains internal to the tube-like structure and does not contact the walls of the passage.

According to some embodiments, an insertion facilitation device comprises the stiffener and an engagement arrangement that releasably engages the stiffener and the tube-like structure. According to some embodiments, the engagement arrangement in an engaged or engaging condition thereof engages the stiffener and the tube-like structure and in a disengaged or disengaging condition thereof releases engagement of the stiffener and the tube-like structure. In these embodiments, with the stiffener and the tube-like structure engaged by the engagement arrangement in its engaged condition, the stiffener facilitates insertion of the tube-like structure into a passage while substantially eliminating axial and or radial movement between the stiffener and the tube-like structure relative to the insertion procedure at hand when properly performed. After insertion of the tube-like structure into the passage, engagement of the stiffener and tube-like structure can be released by the engagement arrangement in its disengaged condition, and the stiffener can be withdrawn entirely or partially from a tube-like structure positioned in a passage. Embodiments of engagement arrangements are disclosed herein

According to some embodiments, the stiffener has a length such that when the proximal portion of the stiffener is engaged with the tube-like structure, the stiffener proximal portion is disposed in a proximal portion of the tube-like structure and the distal portion of the stiffener is disposed in a distal portion of the tube-like structure. According to various embodiments, the insertion device can be configured so that when installed it does or does not longitudinally stretch or stress the tube-like structure. For example, when the insertion device is installed in a catheter having a tip, the proximal end or tip of the stiffener may engage the tip and the distal end of the stiffener may engage the catheter distal end, e.g., via a handle, so as to longitudinally stretch or stress, or not stretch or stress, the catheter. In some embodiments in which it is desired to longitudinally stretch or stress the catheter, at least an engagement arrangement of the insertion facilitation device is provided as a separate part or assembly, e.g., as an insert for a tube-like structure, to be inserted into an otherwise completed tube-like structure, e.g., an otherwise ready for use catheter. Stretching or extending a tube-like structure can facilitate insertion thereof in a passage. In some embodiments in which it is desired not to longitudinally stretch or stress the catheter, an engagement arrangement or a portion thereof including engagement structure is formed with the catheter during manufacture thereof, or in some embodiments, the engagement arrangement comprises an engagement device at least which is provided as a separate part or assembly, e.g., as an insert for a tube-like structure, to be inserted into an otherwise completed tube-like structure, e.g., an otherwise ready for use catheter.

In some embodiments, the length of the stiffener relative to the length of the tube-like structure in which the stiffener extends determines whether the tube-like structure is stretched or stressed when the proximal and distal portions of the stiffener are engaged with the proximal and distal portions of the tube-like structure respectively. In some embodiments, a handle which engages the catheter distal end portion can be adjustably attached to a stiffener so as to make an effective length of the stiffener adjustable, as discussed below.

In some embodiments, the stiffener has a length such that when installed in a tube-like structure, the proximal portion of the stiffener engages the proximal portion of the tube-like structure and the distal portion of the stiffener engages the distal portion of the tube-like structure without the tube-like structure being substantially stretched or stressed. For example, in such an application, the stiffener has a length that is equal to, or substantially equal to, the path the stiffener takes through the tube-like structure from the proximal engagement location to the distal engagement location. In some embodiments, the length of the stiffener is longer than the path the stiffener takes through the tube-like structure from the proximal engagement location to the distal engagement location.

In some embodiments, the location of the distal end portion of the stiffener relative to the distal end portion of the tube-like structure is not fixed. The distal portion of the stiffener according to some embodiments is configured to engage the distal end portion of the tube-like structure via, for example, a handle attached to the stiffener. In some embodiments, the position of the handle (or other feature determinate of the stiffener length) along the length of the stiffener is not fixed. Instead, the axial location of the handle along the stiffener is adjustable. The range of axial adjustment is such that the stiffener is capable of use in a range of tube-like structure lengths and provide the desired degree of stretch or stress, if any, to the tube-like structure.

In some embodiments, in which at least an engagement device portion of an engagement arrangement is provided as a separate part or assembly for use in a tube-like structure in the form of a catheter equipped with a malecot, the stiffener length is selected such that when a proximal portion of the stiffener is engaged with a proximal portion of the catheter and a distal portion of the stiffener is engaged with a distal portion of the catheter, the malecot is stressed such that the arms of the malecot collapse or contract radially, decreasing the overall diameter of the malecot portion of the catheter and increasing the overall length thereof to stress and stretch the catheter.

As indicated above, in use, the tube-like structures are inserted into passages. According to some embodiments, the stiffener is releasably engaged with the tube-like structure at least at or near the proximal end or portion thereof. However, in some embodiments, the stiffener may be engaged at other locations provided that such engagement inhibits or prevents axial and/or angular displacement of the insertion facilitation device with respect to the tube-like structure at least during insertion of the tube-like structure into the passage.

According to some embodiments of the invention, an insertion facilitation device comprises a releasable engagement arrangement to releasably engage the stiffener and a tube-like structure. According to some embodiments of the invention, the releasable engagement arrangement engages the stiffener and the tube-like structure to inhibit or prevent relative motion between the stiffener and the tube-like structure. In some embodiments, the releasable engagement arrangement allows the application of torque to the tube-like structure via the stiffener and the engagement arrangement.

According to some embodiments of the invention, the releasable engagement arrangement comprises an engagement device. In some embodiments, the engagement device comprises a disc-, cylinder- or collar-like engagement device that includes structure that either (a) engages with the interior of a passage in a tube-like structure and releasably engages the stiffener, or (b) releasably engages the passage of the tube-like structure and engages the stiffener. The stiffener and an engagement device can be releasably engaged according to various embodiments of the engagement arrangement, e.g., by a locking wire system, a fastener system, a magnetic system or various mechanical connectors (e.g., luer connectors, detents, spring loaded connectors, press fits, partial turn connectors, e.g. ¼ turn connectors, shot pins, buttons, cams, etc.). An engagement device can releasably engage the passage of the tube-like structure according to various embodiments of an engagement arrangement, and can comprise, e.g., an expandable and contractible structure such as an inflatable balloon, a spring device, a coil, a malecot, etc.

In some embodiments, the engagement device and a part of the stiffener are structured so that one receives the other, e.g., a part of the stiffener is received by the engagement device (or vice versa). In various embodiments, mating structure is provided in the stiffener and the engagement device. In some embodiments, the engagement device comprises an open or closed space or hole therein which receives a part of the stiffener. In one embodiment, the engagement device includes an axial hole or bore which receives the proximal end of the stiffener therein. In another embodiment, at least the proximal end portion of the stiffener includes an axial hole or bore or passage which receives a part of the engagement device therein. The mating structure may releasably engage or be engaged via mechanical structure such as cams, detents, magnetic parts or materials, etc.

According to one embodiment, the engagement device comprises a locking wire system which comprises at least one transverse hole in the engagement device and at least one transverse hole in the stiffener. A stiffener part is received by, e.g., mated with, the engagement device with respective holes in the stiffener and the engagement device aligned or registered with each other. In accordance with one embodiment of the invention, a locking wire is sized to pass through the interior space of the engagement device and the at least one hole therein, and through the at least one hole in the stiffener to cooperate in such a manner that the stiffener and the engagement device are releasably engaged together. For example, in one embodiment, the stiffener may include an interior passage or be hollow, and the locking wire may pass through the interior passage of the stiffener, exit a hole in the stiffener from the interior passage to the exterior thereof, and pass through a hole in the engagement device into the interior space thereof so as to releasably engage the stiffener and the engagement device.

A locking wire may comprise a metallic or non-metallic wire which resists separation of the engagement device and stiffener during insertion of the tube-like structure but which can be withdrawn to allow such separation.

According to some embodiments of a locking system, the stiffener includes two radially directed holes from the interior passage and the engagement device includes two radially directed holes from the interior space. The holes are positioned so that respective pairs of holes are aligned when the stiffener part is mated with the engagement device. In one embodiment, the locking wire may pass from the inside of the stiffener and engagement device, through a pair of aligned or substantially aligned holes in the stiffener and the engagement device to the outside of the stiffener and engagement device, and then through another pair of aligned or substantially aligned holes back into the inside of the engagement device and the stiffener.

In some embodiments of an engagement arrangement that include a locking wire system, the locking wire becomes bent or curved at the transitions between an internal passage or internal space and a hole. The angle of the bends depends upon the angles at which the holes extend, the size and configuration of the wire, and the degree of registration of the sets of holes. This bending and the size and/or strength of the locking wire cooperate to releasably engage the engagement device and stiffener. The bending and wire size are sufficient to hold the stiffener engaged with the engagement device while allowing the locking wire to be withdrawn to allow the stiffener to be disengaged from the engagement device.

In some embodiments of the invention, the engagement device and the stiffener are releasably engaged by a magnetic system. Various embodiments of a magnetic system may comprise cooperating magnetic parts to provide releasable engagement of the stiffener to the engagement device. (Use of the term “magnetic” herein encompasses magnets, magnetic material, magnetizable material, magnetically attracted material such as certain metals, etc.) In various embodiments, magnetic parts may comprise one or more permanent magnets, electromagnets, magnetic material, magnetizable material, magnetically attracted material, etc. In one embodiment, the engagement device comprises a first magnetic part (or component or piece or portion) which is engaged within the tube-like structure in the proximal portion of the structure. A second magnetic part is engaged with a stiffening member in the proximal portion of the stiffening member. Engagement of the stiffener to the tube-like structure is provided by magnetism, and can be released by overcoming, changing, or removing (e.g. in the case of an electromagnet) the magnetism. In various embodiments, the magnetic parts, e.g., the properties and/or configuration thereof, etc., are selected to provide the releasable engagement described herein. The magnetic parts are associated with (e.g., engaged with, attached to, forming part of) the tube-like structure and the stiffener according to various embodiments disclosed herein or evident from the disclosure herein.

In some embodiments, a first magnetic part is engaged with the tube-like structure and a second magnetic part is engaged with the proximal portion of a stiffener. The second magnetic part may be displaced distally by sliding the stiffener away from the proximal portion of the tube-like structure. Resistance to distal displacement of the tubular structure may be provided by maintaining an anchoring apparatus associated with the tube-like structure, such as a malecot or a balloon (as is known in a Foley catheter). The distal displacement of the second magnetic part increases the distance between the first and second magnetic parts. As magnetic force drops dramatically with distance, by moving the second magnetic part an appropriate distance, the stiffening member is effectively disengaged from the catheter, and can therefore be removed. In some embodiments, an anchoring apparatus is not provided, and appropriate selection provides magnetic forces sufficient to hold the stiffener engaged during insertion of the tube-like structure while allowing the stiffener to be withdrawn without displacing the tube-like structure.

In another embodiment, the second magnetic part is slidably disposed with respect to the stiffener and is attached to a metallic or non-metallic wire (or filament) or rod, or actuator which extends through the stiffener or through the tube-like structure towards the distal end. Displacement of the second magnetic part within the stiffener is constrained such that the second magnetic part may extend partially beyond the proximal end of the stiffener but may not fully extend beyond the proximal end of the stiffener. For example, the second magnetic part may comprise a structure or structures, such as a shoulder or an undercut, configured to cooperate with a structure, such as a shoulder or tab, at the proximal portion of the stiffener. In some embodiments the second magnetic part is supported within the stiffener such that rotational displacement of the second magnetic part with respect to the stiffener is limited or prevented. In an embodiment, the proximal portion of the stiffener releasably engages the first magnetic part such that relative rotational displacement between the stiffener and the first magnetic part is limited or prevented. In an embodiment, the proximal portion of the stiffener releasably engages the tube-like structure such that relative rotational displacement between the stiffener and the tube-like structure is limited or prevented. The distal end of the wire or rod is accessible from the distal portion of the tube-like structure. When disengagement is desired, the second magnetic part is moved distally, for example, by grasping and distally displacing the wire or rod, or an actuator, while maintaining the stiffener stationary.

In an alternate embodiment, a mechanical device is included to provide a mechanical advantage to assist in separating the second magnetic part from the first magnetic part. In a non-limiting example, a cam, or cam-like apparatus, is provided in the distal portion of the stiffening member. When separation of the tube-like structure and the magnet affixed to the stiffener is desired, the cam-like apparatus is activated to provide a distal force sufficient to separate the first and second magnetic parts. The force may be applied at the handle or at another location along the length of the stiffener suitable for application of a distally directed force. For example, in an embodiment, a lever comprising a cam shaped surface is provided at a suitable location. The lever is attached to the distal end portion of a metallic or non-metallic wire (or filament) or rod, or actuator. The proximal end portion of the actuator is attached to the second magnetic part. Activation of the lever provides axial displacement of the second magnetic part between at least first and second positions in which the second magnetic part is engaged and disengaged, respectively, with the first magnetic part.

In some embodiments, the engagement device and the proximal portion of the stiffener are provided with engagement surfaces configured to transmit torque from the stiffener to the engagement device. As a non-limiting example in embodiments employing magnetic parts, a first magnetic part is provided with a first engagement surface comprising engagement features configured to cooperate with complementary engagement features on the engagement surface of the second magnetic part. In some embodiments, the engagement surfaces comprise similar but opposite structures configured for engagement resistant to rotational displacement. For example, the engagement surfaces could include one or more pairs of cooperating structures, such as protrusions and recesses, pins and bores, tongues and grooves, tabs and slots, or other pairings of interacting or cooperating structures. Each engaging surface may have one or more of the cooperating structures.

In another embodiment, a magnetic part may be engaged with the proximal portion of the tube-like structure, and an electromagnet engaged with the proximal portion of the stiffener. In one embodiment, the stiffener includes an internal passage through which wires connected to the electromagnet extend to the distal portion of the stiffener.

According to various embodiments of the invention, an engagement arrangement releasably engages at least with either the stiffener or the tube-like structure. Various embodiments of releasable engagement arrangements have been summarized above. An engagement arrangement that releasably engages the stiffener also engages the tube-like structure. Such engagement with the tube-like structure may be releasable or at least substantially non-releasable, at least with respect to a direction of withdrawal of the stiffener.

According to some embodiments, an engagement device comprises engaging structure extending outwardly from the engagement device relative to an axis thereof to engage the passage in the tube-like structure and resist displacement thereof at least substantially more in a first longitudinal direction than in a second, opposite longitudinal direction, and an arrangement that releasably engages the proximal end portion of the stiffener and the engagement device.

According to various embodiments of the invention which include an engagement device that releasably engages the stiffener, in some embodiments the engagement device may be provided as an insert for tube-like structure, and is sized and configured for one way linear displacement within the interior passage of the tube-like structure, e.g., to engage the interior surface of the tube-like structure to allow movement within the tube-like structure in an insertion or first direction and resist or prevent movement in a withdrawal or second direction. According to these embodiments, the engagement device may be moved within the tube-like structure to a desired location, e.g., in a proximal portion of the tube-like structure, from which the engagement device cannot be withdrawn easily or at all. Structure in the tube-like structure may be used to also prevent further advancement and/or withdrawal of the engagement device.

In some embodiments of the invention, the structure of an engagement device that releasably engages the stiffener comprises an exterior surface configured to engage the interior passage of the tube-like structure and to resist or prevent movement in the withdrawal direction. For example, in some embodiments, the outside surface of the engagement device has a barbed surface. In other embodiments, the outside surface of the engagement device may have thin (e.g., vertexed) edges, be toothed, jagged, piniform, spiked, or have other physical features, which allow displacement in the tube-like structure in substantially one direction only, and resist motion in a second opposite direction. According to some embodiments of the invention, the barbs or physical features are flexible or deformable or compliant or retractable when the engagement device is moved in the insertion direction but much less so or not at all for attempted movement of engagement device in the withdrawal direction. In some embodiments of the invention, the engagement device comprises an exterior surface configured to prevent relative rotational movement between the engagement device and the interior passage of the tube-like structure.

According to some embodiments of the invention, an engagement device that releasably engages the stiffener is releasably engaged with the stiffener before the joined stiffener and engagement device are inserted into the tube-like structure. In other embodiments, the engagement device is received within the tube-like structure separate from the stiffener, e.g., as an insert.

According to some embodiments of the invention, structure associated with the tube-like structure is provided to function as an engagement device. The tube-like structure is manufactured or otherwise provided with such structure so that a separate engagement device is not needed to impart engagement device functionality to the tube-like structure. For example, such structure may be integrally or unitarily formed with the tube-like structure, e.g., comprise a suitably configured region of the tube-like structure, or suitably configured material or structure that is permanently bonded or fused to the tube-like structure, etc.

According to some embodiments of the invention, the outside surface of an engagement device that releasably engages the stiffener is adapted to engage internal structure of the tube-like structure. For example, in some embodiments, the engagement device is configured to engage a hole, aperture, eyelet, tab, undercut, screw thread, detent, post, or other structure of the tube-like structure. In some embodiments, the engagement of the engagement device is a permanent, or near permanent, engagement such that relative motion between the engagement device and the tube-like structure is prevented.

Various embodiments of releasable engagement arrangements comprise an engagement device that is releasably engaged with the tube-like structure and engaged with the stiffener. For example, a tube of suitable material, such as Nitinol or spring steel, can be configured to a malecot shape at the proximal portion of the tube. As such, the tube is both the stiffener and the engagement device. A short ring is provided as the actuator to collapse or expand the malecot shape. In one embodiment, the ring sits just distal to the malecot shape while the malecot shape is in its expanded, engaged condition. The ring is substantially coaxial with the tube of suitable material, and of a size that the entire or a portion of the malecot shape can fit within the ring when the malecot shape is in its collapsed, disengaged condition. The ring engages a wire or rod or filament that extends to the distal portion of the stiffener. By moving the wire or rod or filament proximally, the ring is forced over the malecot shape, collapsing it, and changing its state from the expanded, engaged condition to the collapsed, disengaged condition. In another embodiment, the ring sits just proximal to the malecot shape while the malecot shape is in its expanded, engaged condition. By moving the wire or rod or filament distally the ring is forced over the malecot shape, collapsing it, and changing its state from the expanded, engaged condition to the collapsed, disengaged condition. Such engagement with the stiffener may be releasable at least with respect to a direction of withdrawal of the stiffener. According to some embodiments, the engagement device comprises (a) engaging structure that releasably engages the passage and is movable between an expanded condition in which the engaging structure extends outwardly from the engagement device relative to an axis thereof and engages the passage and resists displacement of the engagement device at least longitudinally in the tube-like structure, and a contracted condition in which the engaging structure is retracted and does not engage the passage in the second condition; and (b) an actuator coupled to the engagement device accessible from the distal end portion of the stiffener, activation of the actuator causing the engaging structure to be placed in the expanded condition from the contracted condition and in the contracted condition from the expanded condition.

In some embodiments, the engagement device comprises at least one selectively deformable part configured for selective contraction and expansion thereof inwardly and outwardly relative to a longitudinal axis of the stiffener. In a first condition, the engagement device is contracted inwardly so that it does not engage the passage of the tube-like structure when located therein. In a second condition, the engagement device is expanded outwardly so that it engages the passage of the tube-like structure when located therein. For example, in some embodiments of the invention, the engagement device comprises at least one protruding arm, or at least one coil, or at least one spring, or at least one inflatable balloon, or other expandable and contractible structure. In some embodiments, the engagement device in the first condition thereof does not stress, or does not substantially stress, the tube-like structure, and in the second condition thereof stresses the tube-like structure, which acts to retain the engagement device in place at least during insertion of the tube-like structure in a passage.

The engaging structure according to various embodiments may comprise: a plurality of spring loaded arms and the actuator comprises a rod coupled to the engagement device; an expandable and contractible coil and the actuator comprises a rod coupled to the engagement device; an inflatable and deflatable balloon, and the actuator comprises a valve; a malecot, etc.

According to some embodiments, the stiffener includes a passage extending from a space to the distal end portion of the stiffener, the actuator being disposed in the passage for at least longitudinal movement therein.

According to one embodiment, the engaging structure comprises at least one spring which is normally biased to place the engaging structure in the expanded condition thereof, wherein overcoming the bias places the engaging structure in the contracted condition thereof, and wherein the stiffener includes a closed space in the proximal portion sized to receive therein an engagement device with the engaging structure in the contracted condition thereof and movement of the actuator moves the engagement device out of the space and the spring places the expanding structure in the expanded condition thereof.

In some embodiments of the invention in which a balloon is employed as the engagement device, an inflation valve and, if needed, a fluid conduit are provided for inflating the balloon.

In some embodiments of the invention which include an engagement device that releasably engages the internal passage of the tube-like structure, the engagement device is engaged with the stiffener, e.g., is attached to the stiffener, and can be expanded and/or contracted by an actuator or is movable relative to the stiffener and is attached to an actuator, or is integral with and is formed from the actuator, or is integral with and is formed from the stiffener.

In some embodiments, an engagement device that releasably engages an internal passage in the tube-like structure comprises an expandable part configured to fit in a contracted condition within an internal space of the stiffener, and expands to the expanded condition when moved out of the stiffener. In such embodiments, the actuator may comprise a metallic or non-metallic wire, or filament or rod or actuator, etc. In one embodiment, the rod can be threaded to the engagement device. In another embodiment, the engagement device is formed from the rod such that the rod and engagement device are integral.

In some embodiments, an engagement device that releasably engages an internal passage in the tube-like structure comprises an expandable and contractible coil, or spring arrangement.

According to various embodiments of the invention, an engagement device is located at a proximal end or portion of the stiffener and is adapted to releasably engage the proximal end or portion of the stiffener with the proximal end or portion of the tube-like structure.

In some embodiments, the insertion facilitation device at the distal end or portion of the stiffener comprises a handle. In some embodiments, the handle is attached to the stiffener. In other embodiments, attachment of the handle is adjustable and the handle is slidable relative to the stiffener and is configured to releasably engage the stiffener in a plurality of positions. In some embodiments of the invention, the handle is configured to releasably engage a distal end of the tube-like structure. In some embodiments, the handle is generally conical in shape or a truncated cone or a stepped cone adapted to receive the distal end of a tube-like structure thereon. As discussed above, in an embodiment in which the handle is slidable relative to and engagable with the stiffener, the length of the stiffener can be set by the position of engagement of the handle with the stiffener so as to stretch or stress, or not stretch or stress, the tube-like structure when the stiffener is inserted therein and the handle engaged with the tube-like structure.

In some embodiments, the handle comprises an interior passage sized to accept a wire or filament or rod or other actuator (sometimes referred to herein simply as “wire” or “actuator”). In some embodiments, a handle is attached to the wire or actuator, and this handle mates with the insertion facilitation device or stiffener handle.

According to some embodiments of the invention, the wire or actuator includes a handle at the distal end thereof which is configured to cooperate with the insertion facilitation device handle to releasably engage the wire or actuator to the insertion facilitation device handle.

In accordance with some embodiments of the invention, the insertion facilitation device handle and/or the wire or actuator handle comprises a port to allow aspiration and/or infusion of fluids, or other uses as appropriate, via a connection of a syringe or other device to the port. In some embodiments, the port on the insertion facilitation device handle and/or the wire or actuator handle comprises a threaded connection. In some embodiments, the connection comprises a luer connection or a threaded luer connection.

In some embodiments of the invention, the tube-like structure comprises a catheter. In some embodiments of the invention, the catheter may be a urinary catheter, a coronary catheter or other catheter. In some embodiments of the invention, the tube-like structure comprises a tube used in medical procedures in gastroenterology, proctology, vascular, thoracic, and other medical areas.

According to an embodiment of the invention, a tube-like structure, e.g., a catheter, stiffened with an insertion facilitation device disclosed herein, is inserted into a passage, e.g., the urethra. Proper placement is confirmed by any method known to the medical arts, such as visual confirmation, medical imaging, or, in the case of a drainage catheter, flow of the fluid to be drained. For example, by sizing the stiffener with respect to the passage of the tube-like structure, passage of fluid can serve as an indication of proper placement of the tube-like structure. Further, it allows for the introduction of fluid into the passage or beyond, as may be useful or required. Upon confirmation of proper placement, the insertion facilitation device, including the stiffener, is withdrawn. For insertion facilitation devices including an engagement device that releasably engages the stiffener via a wire or actuator, the wire or actuator is withdrawn from the insertion facilitation device to disengage the engagement device and the stiffener, and after which the stiffener can be withdrawn with the engagement device remaining in the tube-like structure. For insertion facilitation devices including an engagement device that releasably engages the tube-like structure, the locking wire or actuator is worked to contract or otherwise disengage the engagement part. The insertion facilitation device including the engagement device is withdrawn from the tube-like structure.

In some embodiments, the insertion facilitation device is configured to be used with a Foley-type catheter.

A facilitation insertion device according to an embodiment of the invention comprises an elongated stiffener and an engagement device. The stiffener comprises a proximal portion, a proximal end portion and a distal portion and a distal end portion and extends, when installed in a passage in a tube-like structure, along the tube-like structure with the proximal end portion thereof spaced inwardly from an opening to the passage through which the stiffener is inserted into the passage. The stiffener when installed in the tube-like structure provides stiffness or more stiffness to the tube-like structure. The engagement device releasably engages the stiffener in the proximal portion thereof when installed in the tube-like structure and resists displacement of the stiffener in the passage of the tube-like structure in at least one direction when the engagement device engages the stiffener and the tube-like structure.

According to some embodiments, the engagement device is incorporated in the tube-like structure. In some embodiments, the engagement device is a separate structure, and the tube-like structure when formed incorporates the engagement device therein. For example, the engagement device can be placed within the mold used to form the tube-like structure in a process known in the art as insert molding. In the process, the engagement device is placed within a portion of a mold cavity suitable for forming a tube-like structure. The engagement device may be placed in the mold cavity manually or automatically. The mold is closed and a resin is introduced to the mold cavity as is known in the art. The resin flows into the cavity and surrounds the inserted engagement device. Upon removal of the tube-like structure from the mold at the end of the appropriate mold cycle, the engagement device is incorporated within the structure of the tube-like structure.

According to some embodiments, the engagement device comprises engagement structures formed within the tube-like structure, that is, integrally formed as part of the tube-like structure. Such engagement structure cooperates with structure engaged with the stiffener, as discussed herein.

Embodiments comprising an engagement structure formed within the tube-like structure may be used with catheter types known in the art, for example, Foley-type catheters and catheters equipped with malecots. According to some embodiments, insertion facilitation devices in which at least the engagement device is provided as a separate part or assembly (e.g., as an insert) for use with a Foley-type catheter may or may not stretch or stress the catheter, as discussed herein. According to some embodiments, insertion facilitation devices in which all or part of the engagement device or structure is formed with a catheter comprising a malecot do not stress or stretch, or not significantly stretch or stress the catheter.

According to various embodiments of the invention, the insertion facilitation device comprises a handle attached to the distal end portion of the stiffener which engages the tube-like structure with the handle accessible outside the opening to the tube-like structure. According to some embodiments, the actuator extends to the handle and is accessible from the stiffener handle via a handle attached to the actuator.

The invention also provides methods for temporarily increasing the stiffness of a tube-like structure, e.g., a catheter. According to some embodiments, a method comprises inserting a stiffener comprising a proximal portion, a proximal end portion and a distal portion and a distal end portion in the tube-like structure with the stiffener proximal end portion disposed in the catheter proximal portion and the stiffener proximal portion releasably engaged with the passage so as at least to resist withdrawal of the stiffener from the passage; releasing engagement of the stiffener and the passage and withdrawing the stiffener from the tube-like structure.

According to some embodiments, the method comprises releasably engaging the distal portion of the stiffener and the distal portion of the catheter, the releasable engagement of the proximal portion of the stiffener and the releasable engagement of the distal portion of the stiffener existing at a same time.

According to some embodiments, the method comprises releasing engagement of the distal portion of the stiffener and the distal portion of the catheter, wherein engagement of the proximal portion of the stiffener and engagement of the distal portion of the stiffener are released at a same time, and withdrawing the stiffener from the catheter at the same time.

In an embodiment of the inventive insertion facilitation device for insertion of tube-like structures in a passage in a mammalian body, the device comprises an elongate stiffener and an engagement device. The elongate stiffener has proximal and distal ends portions, is adapted to extend in a lumen of a tube-like structure from a distal end portion to a proximal end portion such that the distal end portion of the stiffener is at least partially within the distal end portion of the tube-like structure and the proximal end portion of the stiffener is at least partially in the proximal end portion of the tube-like structure. The stiffener provides stiffness to the tube-like structure greater with the stiffener than without, and is axially displaced within the tube-like structure relative to the tube-like structure.

The engagement device is adapted in an engaging condition to releasably engage the stiffener and tube-like structure with the stiffener extending within the lumen of the tube-like structure such that either or both axial and radial displacement of the stiffener relative to the tube-like structure is substantially eliminated during insertion of the tube-like structure into a body passage. In a disengaging condition, the engagement device is adapted to release engagement of the stiffener and tube-like structure such that the stiffener is at least axially displaceable within the lumen of the tube-like structure and can be withdrawn from the tube-like structure at least so that the proximal end portion of the stiffener in in or adjacent the distal end portion of the tube-like structure.

In some embodiments of the invention, the stiffener comprises a solid or hollow tube, rod, bar, wire, or an assembly of one or more solid or hollow tubes, rods, bars, or wires, and may include a handle adapted to engage or releasably engage the distal end portion of the stiffener.

In some embodiments, the claimed insertion facilitation device comprises an engagement device that is movable between a first condition in which the engagement device releasably engages the stiffener and the tube-like structure, and a second condition responsive to movement releasing the engagement of the stiffener and the tube-like structure.

In another embodiment of the invention, the engagement device is adapted to be inserted into the tube-like structure and engage with the proximal end portion of the tube-like structure.

According to other embodiments of the invention, the engagement device comprises a first outer diameter portion configured to engage a portion of the lumen at the proximal portion of the tube-like structure and at least substantially prevent at least one of radial motion and axial motion in a distal direction with respect to the lumen, and a portion of the engagement device engages at least a portion of the proximal end portion of the stiffener.

In some embodiments of the invention, the engagement device comprises a second outer diameter portion smaller than the first outer diameter portion, an inner bore in at least the second outer diameter portion, and at least one radial opening between the inner bore and the second outer diameter portion. The stiffener comprises an outer surface, an axial bore extending from the distal end portion toward to proximal end portion, at least one opening between the inner bore and the outer surface, with the opening configured to align with the at least one radial opening in the engagement device. Embodiments of the inventive insertion facilitation device may include a locking wire adapted for axial movement within the axial bore of the stiffener from the distal end portion to the at least one pair of aligned radial openings, the locking wire extending at least partially through the aligned openings and releasably engaging the engagement device and the stiffener. In some embodiments, the distal end of the locking wire comprises a handle engaged with the locking wire.

In an embodiment, the engagement device is engaged with the proximal end portion of the stiffener and is adapted to releasably engage the lumen of the tube-like structure, a structural feature of the tube-like structure, or one or more openings in the tube-like structure. The engagement device may include a selectively expandable resilient spring arm or a selectively expandable malecot according to some embodiments.

According to an embodiment of the invention, the tube-like structure comprises a first element of the engagement device and the stiffener comprises at least a second element of the engagement device adapted to releasably engage the first element. In an embodiment, the first element is a magnetic material and has a first interface surface and the second element is a second magnetic material having a second interface surface and an appropriate polarity to create a magnetic attraction with the first magnetic material sufficiently strong to engage the first and second interface surfaces. In some embodiments, the first interface surface includes features configured to cooperate with features on the second interface surface to resist relative angular motion between the first and second magnetic materials. The features, according to an embodiment, are a projection or recesses on the first surface and a recess or projection on the second surface suitable to engage the first element. The projection and recess may comprise mating partial helical thread forms, or respective halves of a luer fitting.

In another embodiment, the second magnetic material is slidably disposed with respect to the proximal portion of the stiffener and may be selectively advanced proximally to engage with the first magnetic material or distally to disengage with the first magnetic material. According to some embodiments, the selective advancement and retraction of the second magnetic material is effected by an actuator configured for axial displacement within the axial bore of the lumen.

In another embodiment according to the present invention, a method for providing stiffness to a tube-like structure during insertion into a passage comprises introducing a stiffener to a lumen of a tube-like structure through an opening in the distal end portion of the structure, advancing the stiffener axially toward the proximal portion of the tube-like structure, and actuating an engagement device to releasably engage directly or indirectly the stiffener to a selected portion of the tube-like structure lumen.

In an embodiment of the invention, a catheter system for facilitating insertion of a catheter into a body passage of a mammal comprises a catheter, an elongate stiffener, having a proximal end portion and a distal end portion, and extendable into the catheter from the distal end portion to the proximal end portion of the catheter, with the distal end portion of the stiffener at least partially within the distal end portion of the catheter and axially displaceably within, and relative to, the catheter, and an engagement device releasably engaging the stiffener and the catheter with the stiffener extending within the catheter such that either or both relative axial and radial displacement of the stiffener relative to the catheter is substantially eliminated and when the stiffener and catheter are disengaged, the stiffener is at least axially displaceable within the catheter and can be withdrawn from the catheter at least to the extent that the proximal end portion of the stiffener is in or adjacent the distal end portion of the catheter, wherein at least part of the engagement device is movable, the engagement device in a first condition releasably engaging the stiffener and the catheter, and in a second condition, responsive to movement, releasing engagement of the stiffener and the catheter. The engagement device may engage or releasably engage a portion of the catheter according to embodiments of the invention. In another embodiment, the engagement device is releasably engaged to the proximal end portion of the stiffener and is adapted to be axially displaced within and relative to the catheter to a proximal portion of the catheter and engage a proximal portion of the catheter.

Various embodiments of insertion facilitation devices and tube-like structures are described herein. Other embodiments will be apparent to those of skill in the art from the disclosure herein, and are intended to be encompassed within the overall disclosure herein.

### BRIEF DESCRIPTION OF THE DRAWINGS

The following detailed description, given by way of example and not intended to limit the invention to the disclosed details, is made in conjunction with the accompanying drawings, in which like references denote like or similar elements and parts, and in which:

FIG. 1 is a side view of an insertion facilitation device according to one embodiment of the invention which includes a stiffener and an engagement device that releasably engages the stiffener;

FIG. 2 is an enlarged side view of the proximal portion of the insertion facilitation device depicted in FIG. 1 also showing an embodiment of the engagement device;

FIG. 3 is a plan view of the insertion facilitation device proximal end portion depicted in FIG. 2 also showing an embodiment of the engagement device;

FIG. 4 is an axial sectional side view of the insertion facilitation device proximal end portion depicted in FIG. 3;

FIG. 5 is a side view of a catheter which includes an insertion facilitation device according to FIGS. 1-4;

FIG. 6 is a plan view of the proximal end portion of the catheter of FIG. 5;

FIG. 7 is an axial sectional view of the proximal end portion of the catheter depicted in FIG. 6 taken along line 7-7 and showing the proximal end portion of an embodiment of the insertion facilitation device;

FIG. 8 is a side view of an insertion facilitation device according to another embodiment of the invention which includes a stiffener and an engagement device that releasably engages a catheter;

FIG. 9 is an enlarged view of the insertion facilitation device proximal end portion depicted in FIG. 8 showing the engagement device in an expanded condition;

FIG. 10 is an enlarged plan view of the insertion facilitation device proximal end portion depicted in FIG. 8 with the engagement device in contracted condition and fully within the stiffener;

FIG. 11 is an axial section view of the insertion facilitation device proximal end portion depicted in FIG. 10, taken along line 11-11;

FIG. 12 is an enlarged plan view of the proximal end portion of a catheter;

FIG. 13 is an axial sectional view of a catheter according to FIG. 12 taken along line 13-13 including a stiffener showing cooperating magnetic parts associated with the proximal end portion of the catheter and stiffener according to an embodiment of the invention;

FIGS. 14A-14C are views of the proximal end portion of the catheter depicted in FIG. 13 showing engagement surfaces of cooperating magnetic parts according to various embodiments of the invention;

FIG. 15 is an enlarged view of the proximal end portion of a catheter similar to that in FIG. 13 showing a stiffener proximal end portion therein and cooperating magnetic parts according to another embodiment of the invention;

FIG. 16 is a perspective view of an engagement device and the proximal end portion of a stiffener according to an embodiment of the invention;

FIG. 17 is a cross sectional view of the engagement device and stiffener of FIG. 16 taken along line 17-17;

FIG. 18 is a perspective view of an engagement device according to an embodiment of the invention; and

FIG. 19 is a cross sectional view of the proximal end portion of a stiffener engaged with the proximal end portion of a tube-like structure according to an embodiment of the invention.

### DETAILED DESCRIPTION OF PREFERRED EMBODIMENTS

Embodiments of the invention are described below with reference to the accompanying drawings which depict embodiments of insertion facilitation devices and catheters. However, it is to be understood that application of the invention encompasses other tube-like structures and is not limited to catheters, and to insertion facilitation devices other than those illustrated. Also, the invention is not limited to the depicted embodiments and the details thereof, which are provided for purposes of illustration and not limitation.

Insertion facilitation devices (or tools) according to embodiments of the invention provide stiffness to a tube-like structure, as discussed above, and a range of stiffnesses can be provided. As discussed above, such stiffness can facilitate insertion of a tube-like structure into a passage. However, it should be understood that the particular stiffness which the device is to provide may depend upon, e.g., the particular application, tube-like structure e.g., catheter and/or subject. Upon successful insertion, the tube-like structure remaining in the passage may be supported by the passage and the stiffener may be withdrawn from the tube-like structure.

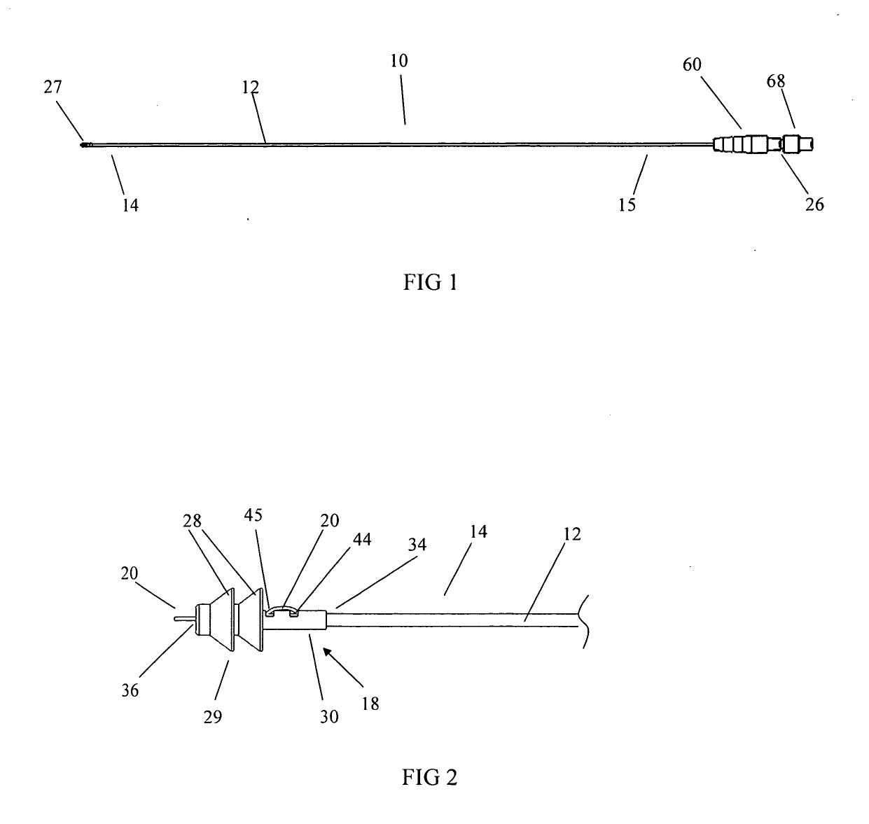

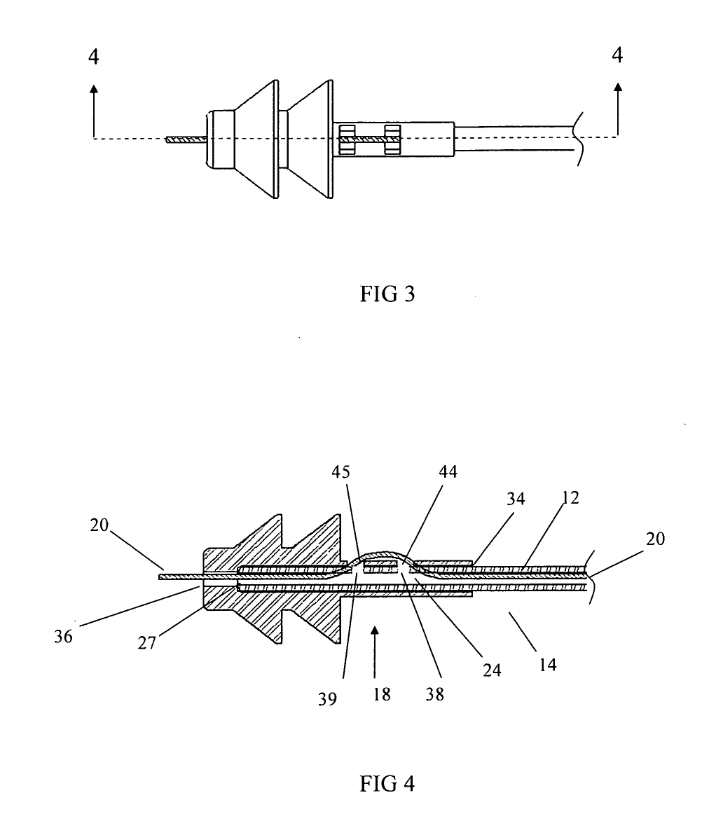

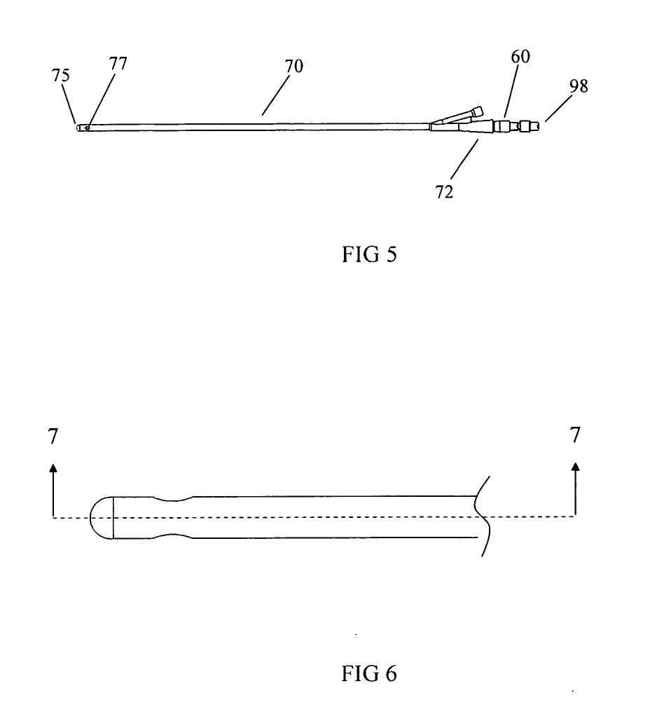

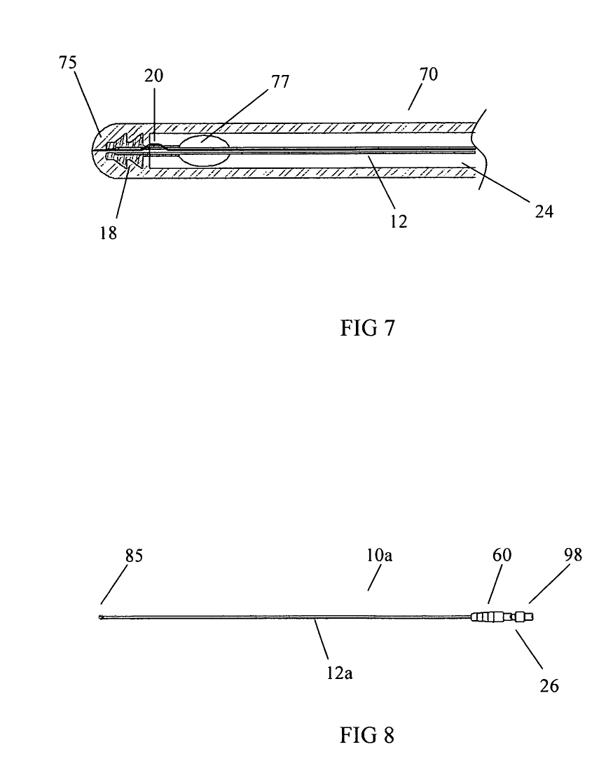

1001031 Referring to FIG. 1, a catheter insertion device or tool 10 according to an embodiment of the invention comprises a stiffener 12, which may be implemented, e.g., by a solid or hollow tubular member. The stiffener 12 also may be referred to as a pusher. The stiffener 12 comprises a proximal end portion 14 and a distal end portion 15. According to various embodiments of the invention, releasable engagement of the stiffener within a catheter (or other tube-like structure) is achieved via an engagement device 18 (FIGS. 2 and 3) located within the catheter that is prevented from moving relative to the catheter at least during insertion of the catheter and a releasable engagement of the stiffener 12 and the engagement device 18, as discussed above. The engagement device 18 engages the lumen (e.g. 71 in FIG. 7) in the catheter such that the stiffener 12 cannot damage the catheter or exit therefrom during the insertion procedure, but rather remains captured by the engagement device until purposely released. The insertion facilitation device remains internal to the tube-like structure and does not contact the walls of the passage. This reduces risk of damage to the passage into which the catheter is inserted and to the catheter.

According to various embodiments, the engagement device 18 comprises a disc-or cylinder-like member including an internal space in the form of an axially extending bore or hole 34 and structure 28 (frustro-conically shaped in the illustrated embodiment) projecting outwardly for engaging the catheter's lumen.

As discussed above, a catheter or other tube-like structure may be provided with an engagement device 18 (or include structure that releasably engages a stiffener) that is integrally formed within the catheter or otherwise attached during manufacture thereof, etc. In the embodiment illustrated in FIGS. 1-4, the engagement device 18 is not integrally formed with the catheter, and is provided as a separate piece or insert to be inserted into a catheter during or after formation thereof. For example, the engagement device 18 releasably engaged with the stiffener may be inserted within a catheter before the end of manufacture thereof, or at some point in the distribution chain, or when being readied for use.

In the embodiment depicted in FIGS. 1-4, the engagement device 18 can be supplied assembled and engaged with the stiffener 12, with the insertion facilitation device inserted in the catheter which is ready for insertion, or supplied unassembled. FIGS. 1-4 show a fully assembled insertion facilitation device 10, or a portion thereof, including the engagement device 18.

As illustrated in FIGS. 2-4, according to some embodiments of the invention, the stiffener 12 can be releasably engaged with the engagement device 18 by a locking wire or filament 20. According to some embodiments, as illustrated in FIGS. 1-4, the stiffener 12 includes an internal passage 24 (FIG. 4) extending therein from the proximal end portion 14 to the distal end portion 15 (FIG. 1) and proximal and distal openings 26 (FIG. 1), 27 (FIG. 4), respectively, therein to the internal passage 24. In an embodiment, the openings 26, 27 may be spaced and located at or near opposite ends of the passage, or in other embodiments located elsewhere. The locking wire 20 is inserted into the internal passage 24 of the stiffener through an opening, e.g. 26, in the distal portion of the stiffener. The locking wire 20 in some embodiments includes a locking wire handle 68 (FIG. 1) attached to the distal end of the locking wire which mates with a handle 60 (FIG. 1 and discussed below) attached to the stiffener 12. For use in catheters in which the engagement device 18 may prevent or restrict flow into the catheter lumen 71 (FIG. 7), the engagement device may include an opening 36 (FIG. 4) and a passage, e.g. axial bore 34, through the engagement device. The internal passage 24 in the stiffener 12 and the opening 26 are provided in these embodiments for the locking wire to pass through, and are sized to allow movement of the locking wire therethrough. In one embodiment, the stiffener 12 may comprise a hollow tube including an internal passage 24 that extends the entire length of the stiffener 12.

The engagement device 18 is configured to releasably engage the stiffener 12 and to be engaged within a catheter lumen 71 at a desired location. In the embodiment depicted in FIGS. 1-4, the engagement device 18 includes larger and smaller outer diameter portions 29, 30 and the internal passage or bore 34 axially extending therein. The internal passage 34 is open on the end of the engagement device nearer the distal end of the insertion facilitation device 10 and extends axially for a sufficient distance at least in the smaller diameter portion 30 to receive therein the proximal end of the stiffener 12. Alternatively, the smaller diameter portion 30 could be of a size to fit within the internal passage 24 of the stiffener 12. Depending upon the type of catheter with which the insertion facilitation device is to be used, the internal passage 34 may or may not extend entirely through the engagement device. For example, the internal passage 34 may be blind and terminate within the engagement device 18, or may extend entirely through the engagement device and terminate in an opening 36, as depicted in FIGS. 2 and 3.

The diameter of the internal passage 34, at least in the smaller diameter portion 30, and the outer diameter of the stiffener 12 are sized so the stiffener 12 is received in the engagement device 18 such that they can be easily separated. The larger diameter portion 29 is configured so that the engagement device 18 may be engaged in the catheter lumen 71, as discussed in more detail below.

According to some embodiments, one or more holes are provided in the stiffener 12 extending outwardly, transversely and preferably radially, from the internal passage 24 through the wall of the stiffener to communicate the internal passage 24 with the exterior of the stiffener 12 in the proximal end portion 14 of the stiffener 12. In these embodiments, one or more holes are also provided in the engagement device 18 extending outwardly from the internal passage 34 through the wall of the engagement device. These holes are sized to permit passage therethrough of the locking wire 20 to releasably engage the stiffener 12 and the engagement device 18. In some embodiments, one or more holes in the stiffener and one or more holes in the engagement device are registered, i.e., aligned, when the engagement device receives the stiffener, as shown in FIGS. 2-4. According to one embodiment, as depicted in FIGS. 2-4, two such outwardly extending holes 38, 39 are provided in the stiffener 12 and two such outwardly extending holes 44, 45 are provided in the engagement device 18. The holes 38, 39 and 44, 45 are sized to allow passage therethrough of the locking wire 20. The engagement device 18 is disposed on the stiffener 12 so that holes 38 and 44 are registered (aligned), and holes 39 and 45 are registered.

According to some embodiments, as indicated above, the internal passage 34 in the engagement device 18 may terminate therein, i.e., not extend to the exterior of the engagement device at the proximal portion of the stiffener. In these embodiments, the locking wire 20 terminates in the engagement device 18. In other embodiments, the internal passage 34 in the engagement device 18 extends completely therethrough, communicating the internal passage 24 of the stiffener 12 with the proximal end of the stiffener. In these embodiments, when the engagement device 18 is positioned in a catheter, and the stiffener is removed, a passage is provided through the engagement device in the lumen of the catheter.

The stiffener 12 is releasably engaged with the engagement device 18 according to the embodiment depicted in FIGS. 2-3, as follows. With the engagement device 18 positioned on the stiffener proximal portion 14 with holes 38 and 44 registered and holes 39 and 45 registered, the locking wire 20 passes through the internal passage 24 in the stiffener 12 from the distal end portion 15 thereof to the proximal portion 14 thereof, through aligned holes 38 and 44 from the internal passage 24 of the stiffener 12 and the internal passage 34 of the engagement device 18 to the outside of the engagement device 18 and stiffener 12 and then through aligned holes 45 and 39 from the outside of the engagement device 18 and stiffener 12 back into the internal passage 24 of the stiffener 12. The locking wire 20 becomes bent or curved at the transitions between an internal passage and a hole. The angles of the bends depends upon the angles at which the holes extend and the degree of registration of sets of the holes. The bending and locking wire 20 size are sufficient to hold the stiffener 12 in engagement with the engagement device 18 while allowing the locking wire 20 to be withdrawn to disengage the stiffener 12 and the engagement device 18 and the stiffener 12 thereafter may be withdrawn from the catheter. For example, a thin (e.g. approximately 0.002-0.020 inch diameter) wire made of, e.g., Nitinol, is known to have sufficient tensile strength to withstand anticipated forces, and yet is small enough to fit through appropriately sized holes 38, 39, 44, and 45. It will be understood by those skilled in the art that other sizes and material choices of the locking wire could also suffice. According to some embodiments, one or both of the sets of holes 38, 44 and 39, 45 extend radially or more radially than axially from the respective internal passage. This acts to increase the angle of the bends in the locking wire and thereby increase the engaging action thereof. Other embodiments (not shown) of the invention require only one pair of aligned holes, e.g. 38, 44, and the locking wire 20 to achieve an acceptable engagement as would be understood by one skilled in the art.

According to some embodiments, assembly of the insertion facilitation device 10 includes manually passing the locking wire through the aligned sets of holes. In other embodiments, this process can be automated. This can be done at the time of use, in distribution, or prior to the end of manufacture. As discussed herein, the engagement device 18 may be provided as an insert to be inserted into a catheter after manufacture of the catheter itself, or engaged with or forming part of a catheter during manufacture.

Various embodiments of engagement devices may be provided that permit the engagement device to be inserted into a catheter and moved to the desired final location and engaged thereat at least with respect to withdrawal thereof. Such structure may comprise projections extending outwardly from the engagement device which are configured and/or flexible or deformable to permit one way movement of the insert through a passage of the catheter, e.g., similar in operation to an arrowhead.

In an embodiment of the invention depicted in FIGS. 2 and 3, engagement device 18 comprises one or more projections 28 extending outwardly from the longitudinal axis of the engagement device. The projections 28 are configured to slope inwardly from a radially extending edge in the direction of movement, e.g., extend frustro-conically in the direction of movement. The projections are sized to be slightly larger than the diameter of a catheter's lumen 71 so as to contact the inner wall of the lumen. Sloped projections facilitate displacement of the engagement device 18 in the lumen 71 in contact with the inner wall thereof in the direction of slope of the projections, i.e., in an insertion direction. In one embodiment, the projections may be rigid and therefore temporarily enlarge the diameter of the lumen 71 as the engagement device 18 is moved therein, with the diameter of the lumen 71 contracting to or near to its original diameter when the engagement device no longer contacts that portion of the lumen inner wall. The contracted lumen diameter and the radial edge of the engagement device substantially resist or prevent displacement of the engagement device in a direction away from the sloped direction, i.e., a withdrawal direction.

In some embodiments, the engagement device projections 28 may be flexible, resilient or deformable so as to be radially expandable and contractible, with the projections being biased outwardly into contact with the inner wall of the catheter's lumen 71. Biasing the flexible projections 28 outwardly urges them into contact with the inner wall of a catheter's lumen 71. The projections are configured to flex inwardly and contract with engagement device displacement in one direction only (the insertion direction) or to flex more easily in the insertion direction, and resist deflection if advanced in the withdrawal direction. For example, while the engagement device 18 is advancing in the lumen 71 in the insertion direction towards the proximal portion thereof, the engaging structure of the engagement device is forced inwardly. However, when the engagement device 18 is forced in the opposite withdrawal direction, the engagement structure 28 engages the lumen 71 to resist such movement. For example, referring to FIGS. 2-4, the engaging structure 28 is sloped in the insertion direction, but includes a thin or vertexed edge 32 that engages the lumen 71 in the withdrawal direction. Many other structures that operate in that or a similar manner, or operate to flex in one direction (e.g., the insertion direction) and not in the opposite direction, to provide one-way movement of a device in a tube will be evident to those of skill in the art.

In some embodiments, the engagement device projections 28 are also configured to prevent angular displacement of the insert, as well as axial displacement, to facilitate the application of torque to the insertion device. Such configuration may include circumferentially extending structure and/or a strong engagement of the projections with the inner wall of the catheter's lumen 71. For example, such structure may include barbs, or toothed, jagged, piniform, spiked, or other structure.

In some embodiments of the invention, the distal end portion 15 of the insertion facilitation device 10 comprises a handle 60 secured to the distal end of the stiffener 12, as shown, for example, in FIGS. 1 and 5. According to various embodiments of the invention, the handle 60 can comprise any suitable shape, e.g., a generally conical, frusto-conical, or a stepped cone shape, and include an internal axial bore or passage (not shown) having a diameter suitable to receive the stiffener 12 therein and be attached to the stiffener 12 using any suitable technology, e.g., bonding, fusion, adhesives, etc., fasteners (e.g., a set screw), a clamp, an adjustable collar, a crimp engagement, opposing portions of the pusher handle compressing the element between the portions, or other suitable configurations. Depending upon the application, the internal passage can extend through the handle 60.

According to various embodiments of the invention, insertion facilitation devices 10 may be provided unassembled, partially assembled or fully assembled. According to some embodiments, the stiffener 12 and the handle 60 are provided separately for assembly during distribution or at the time of use. In some such embodiments, the handle 60 may be configured to be adjustably attached to the stiffener 12 and the handle 60 attached to accommodate different types or lengths of catheters, or for purposes of stretching or stressing, or not stretching or stressing a catheter when the stiffener 12 is deployed and engaged. In such embodiments, the engagement device 18 and the stiffener 12 may be provided assembled releasably engaged together or separately for later assembly. In such embodiments, the insertion facilitation device 10 may be provided in a fully assembled condition.

In embodiments in which the stiffener 12 and the handle 60 are provided assembled together, and in embodiments in which the insertion facilitation device 10 is provided in a fully assembled condition, stiffeners of various lengths and insertion devices of various lengths may be provided to accommodate a wide range of catheter-types and lengths, and for purposes of stretching or stressing, or not stretching or stressing a catheter when the stiffener is deployed and engaged.

One application of the insertion device depicted in FIGS. 1-4 is for use with a prior art Foley-type urinary catheter, e.g. 70 (FIG. 5). A Foley-type catheter comprises a flexible tube comprising an interior passage or lumen 71 extending from one end (the proximal end) to the opposite end (the distal end). In use, the Foley-type catheter 70 may be passed through the urethra with its proximal end terminating in the bladder and its distal end extending beyond the meatus. A function of the Foley-type catheter is to drain urine from the bladder through the lumen of the catheter. Typically, a Foley-type catheter comprises an inflatable balloon in the proximal portion. In use, with the catheter inserted with the balloon located in the bladder, the balloon is inflated to maintain the catheter in the bladder and prevent unintended movement of the catheter. A Foley-type catheter equipped with a balloon includes an inflation passage or lumen extending from beyond the meatus and terminating in the balloon for inflating it. This inflation passage is fitted with a port or valve in or near the end of the catheter extending beyond the meatus for controlling inflation and deflation of the balloon. A Foley-type catheter typically also includes holes or eyelets 77 (FIG. 5) in or near the proximal end thereof which extend from the lumen and through the side wall of the catheter to allow the bladder contents to drain from the bladder into the lumen of the catheter. An example of a Foley-type catheter is described in U.S. Pat. No. 6,602,243, the entire disclosure of which is incorporated herein by reference.

FIGS. 5 and 7 illustrate an insertion facilitation device 10 according to an embodiment of the invention installed in a Foley-type catheter 70. The handle 60 of the insertion facilitation device is engaged with the distal end 72 of the catheter 70 and with the distal end of the stiffener (not shown). The stiffener 12 has a length such that when the proximal portion of the stiffener is engaged with the engagement device 18 located at the catheter tip 75 and the handle 60 extends from the catheter 70 in engagement with the distal end 72 a distance sufficient to permit the catheter to extend its full length unstressed or slightly stressed, and for the handle to be grasped and manipulated. As mentioned above, in some embodiments of an insertable engagement device, the insertion facilitation device may releasably stretch or extend the catheter. In some of those embodiments, the catheter may comprise a malecot (not shown) attached to the proximal portion thereof as described, for example, in cited WO 2008/067557.

As discussed above, a Foley-type catheter includes one or more eyelets 77 located in the proximal portion of the catheter in communication with the lumen 71 to permit fluid to enter the lumen 71 of the catheter 70 and be drained therethrough. The eyelets 77 are located spaced from the tip 75, as depicted in FIG. 7. In the illustrated embodiments, the engagement device 18 is located in the catheter 70 beyond the eyelets 77 in the tip 75. However, in other embodiments, the engagement device can be located on the distal side of the eyelets.

In another embodiment, an insertion facilitation device is capable of interacting with a feature on a tube-like structure. For example, as mentioned, a Foley-type catheter typically includes eyelets 77 in the proximal end portion. An engagement device can be configured to engage, e.g., to enter into, one or more of the eyelets. According to one embodiment, the engaging structure can be outwardly biased such as to normally extend beyond the inner diameter of the lumen. The engagement device 18 is inserted into the lumen 71 with the engagement structure, e.g., protrusions, retracted. Upon reaching the eyelets 77, the engaging structure expands, effectively locking the stiffener to the lumen through the walls of the eyelets.

FIGS. 8-11 depict another embodiment of an insertion device 10a which includes a stiffener 12a and an expandable engagement device 85. The stiffener 12a includes an internal passage 24 (FIG. 11), generally as described for stiffener 12, and has openings 26 (FIGS. 8) and 27 (FIG. 11) to the internal passage 24 in the distal and proximal portions. The engagement device 85 comprises a spring (also referenced by 85) which includes two or more oppositely biased spring arms 94. According to one embodiment, the spring arms comprise c-shaped leaf springs joined at opposite ends thereof. In other embodiments, the spring arms may be parts of a basket or hoop-type spring. Other non-limiting examples of suitable shapes for deformable members include a radially protruding arm or arms, coils, springs, sinusoidal forms, or other shapes that can be contracted radially. In one embodiment, the engagement device may comprise an inflatable balloon.

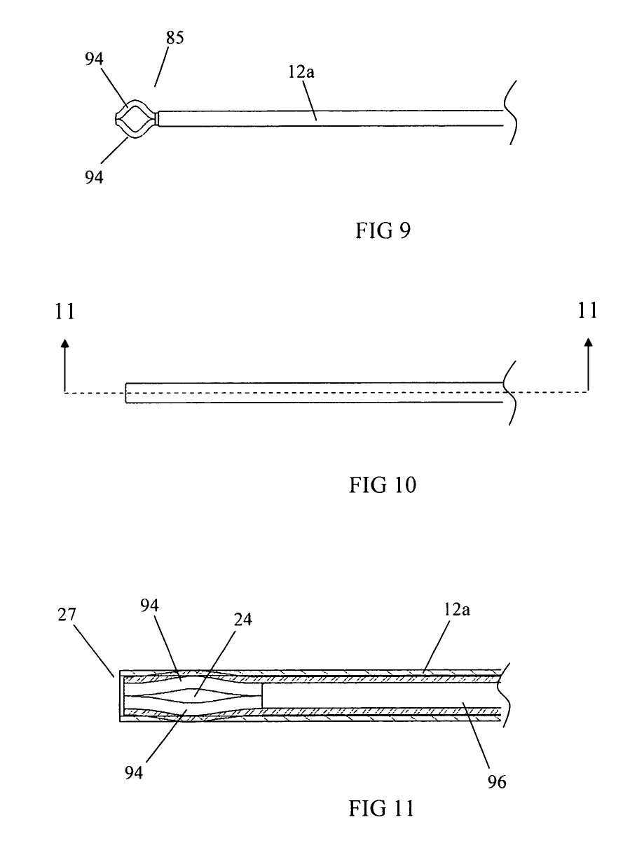

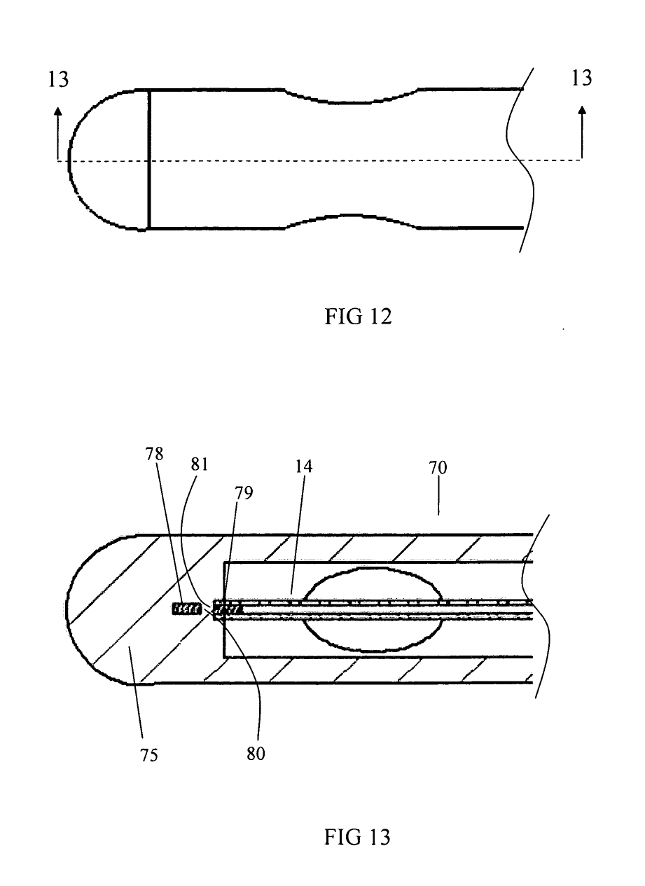

Referring to FIGS. 8-11, in the normal condition of the spring 85 (FIG. 9), the spring arms are outwardly, e.g., radially, extended with the central regions of the spring arms spaced apart, and in a compressed condition (FIG. 11), the spring 85 is contracted with the spring arms 94 restrained and central regions thereof substantially parallel to each other with little or no space therebetween.

The engagement device 85 is attached to a rod 96 (FIG. 11) that extends in the stiffener internal passage 24 from the proximal portion, where it is attached to the engagement device 85, to the distal portion where it exits opening 26 and is accessible for use in activating and deactivating the engagement device 85, e.g., connected to a handle 98. The rod 96 and the engagement device 85 may be attached in any suitable manner, e.g., the rod 96 may be threaded to the engagement device 85. The rod 96 and the engagement device 85 may also be of one piece, where the engagement device 85 is formed and trained out of the rod stock, provided the rod 96 is made of a suitable material such as, for example, Nitinol. In the contracted condition (FIG. 11), the spring 85 may be located in the internal passage 24 of the stiffener 12a, which restrains the spring arms 94. In the expanded condition (FIG. 9), the spring 85 is located outside of the stiffener 12a, having exited through opening 27 with the spring arms 94 unrestrained and expanded. The rod 96 is used to push and pull the engagement device 85 in the internal passage 24 of the stiffener 12a. The rod 96 is slidably disposed within the internal passage 24, and is attached to the actuator handle 98 at the distal end. The rod 96 is of a length that when the actuator handle 98 is moved forward toward or mated with the stiffener handle 60, the spring 85 will be outside of stiffener 12a. When the actuator handle 98 is withdrawn from the stiffener handle 60, the spring 85 is pulled into the stiffener.

The insertion device 10a may be assembled according to one embodiment as follows. The rod 96 is provided with the engagement device 85 located at the proximal portion thereof. The distal end of the actuator rod 96 is inserted into the internal passage 24 of the stiffener 12a through an opening, e.g. 27 and pushed until the distal end exits the distal portion of the stiffener 12a through an opening, e.g. 26. The spring 85 is compressed and pulled into the internal passage 24 of the stiffener 12a by pulling the distal end of the rod 96 or the actuator handle 98 attached thereto relative to the stiffener 12a. Alternatively, the spring 85 can be compressed and pushed into the internal passage 24 of the stiffener 12a through opening 26 in the distal portion of the stiffener. Then, the rod can be pushed into the stiffener until the spring 85 is located adjacent to opening 27.

A handle 98 may be attached to the distal end of the rod 96 in any suitable way. The length of the stiffener element and the rod are determined generally as described above.

The insertion device 10a is inserted into a catheter as follows. With the engagement device 85 located in the internal passage 24 of the stiffener 12a, the stiffener is inserted through a catheter distal end until the proximal end of the stiffener 12a reaches the desired location within the catheter, for example, the proximal portion of the catheter. The actuator rod 96 is then pushed relative to the stiffener 12a to push the engagement device 85 out of the stiffener 12a. That releases the spring arms 94 which spring into engagement with the inner wall of the catheter lumen 71. In this condition (FIG. 9), the spring arms 94 engage the lumen inner wall sufficiently to prevent or substantially prevent relative axial and/or radial displacement between the device 85 and the catheter. Alternately, once the insertion facilitation device 10a is positioned, the stiffener 12a is retracted while holding the rod 96 stationary, to release the engagement device 85 from the internal confines of the stiffener 12a. The spring arms 94 assume their natural biased state and can engage the lumen inner wall as described above.

The stiffener 12a is removed from a catheter as follows. The rod 96 is pulled relative to the stiffener 12a to withdraw the engagement device 85 out of engagement with the lumen wall into the internal passage 24 of the stiffener 12a. Alternately, the engaging device 85 can remain fixed and the stiffener 12 advanced to move the spring arms 94 to a radially compressed position. The stiffener may now be withdrawn from the catheter.

In some embodiments, the insertion facilitation device includes cooperating magnetic parts associated with the engagement device and the stiffener. FIGS. 13-15 illustrate some of these embodiments.

Referring to FIG. 13, the engagement device comprises a first magnetic part 78 located in the proximal portion of the catheter, e.g., in or near the proximal tip 75. The magnetic part 78, in some embodiments, may be formed with the catheter during manufacture thereof, as described above. In other embodiments, the magnetic part 78 may be attached to an engagement device similar to the one shown in FIGS. 2, 3 or 4. The magnetic part 78 may be attached to the distal end of an engagement device 18 shown in FIGS. 2 and 3, but without the locking wire structure.

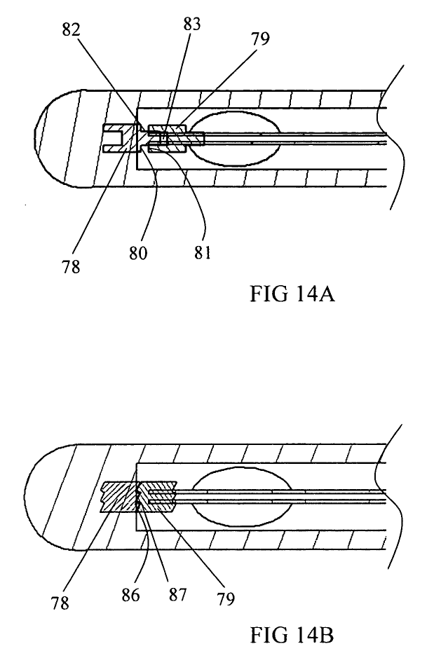

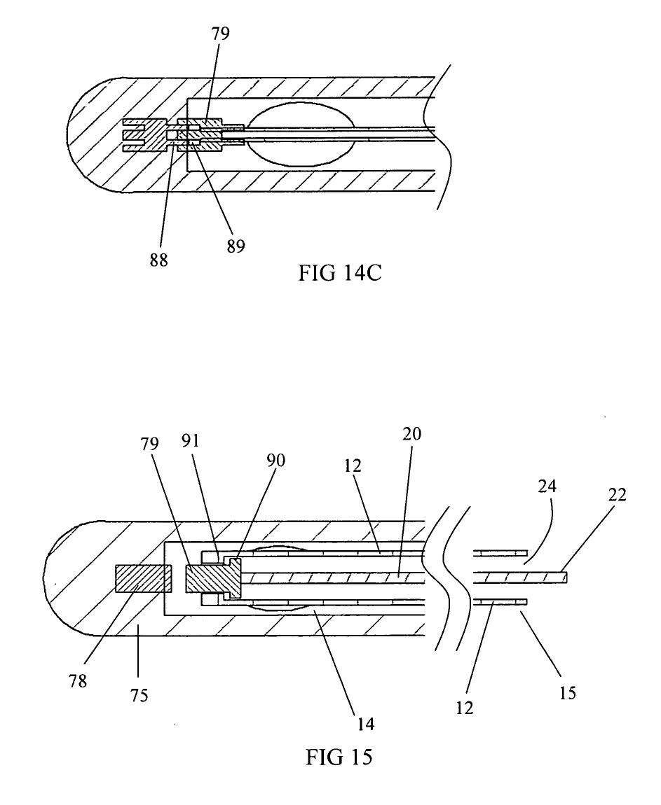

Referring to FIG. 13, the stiffener proximal portion 14 comprises a suitably configured second magnetic part 79 (FIGS. 13 and 14) which cooperates with magnetic part 78 to provide releasable engagement of the stiffener 12 to the catheter 70. According to some embodiments, the magnetic part 79 is fixed in the interior passage or to the proximal portion of the stiffener 12, e.g., by adhesive, or a mechanical action, e.g., crimping, or any suitable way. In some embodiments, the proximal portion of the stiffener, or the entire stiffener, may be made of magnetic or magnetizable material configured to exert a magnetic force suitable for the purposes described herein.

The magnetic parts 78, 79 according to one embodiment of the invention have interface surfaces (respectively 80, 81) which comprise cooperating features which resist relative angular movement between the first and second magnetic materials 78, 79. Non-limiting illustrative examples of suitable interface surfaces are shown in FIGS. 14A-14C. (For clarity, magnetic parts 78, 79 are shown slightly separated in FIGS. 14A-14C). For example, the first magnetic part 78 may comprise a projection 82, such as a tongue or tab, sized and shaped to be received within a recess 83, such as a groove or slot, on the interface surface 81 of the second magnetic part 79. According to one embodiment, the interface surfaces 80, 81 comprise irregular features 86, 87 in mating pairs configured to prevent, or at least limit, angular movement between first and second magnetic parts (78, 79). In one embodiment, the interface surfaces comprise one or more pins or posts 88 (FIG. 14C) suitably sized, shaped, and located to be received in one or more recesses 89 on the second magnetic part 79. Pins/posts 88 and recesses 89 may be interchangeably located on either the first magnetic part 78 or the second magnetic part 79 in mating pairs.

According to some embodiments of the invention, the second magnetic part 79 may be slidably disposed within at least the proximal portion 14 of stiffener 12, as illustrated in FIG. 15, and constrained such that the second magnetic part 79 may extend to, or only partially beyond, the proximal end of the stiffener 12. According to an embodiment of the invention, the second magnetic part 79 may comprise a first structure, such as first shoulder 90, configured to cooperate with a second structure at the proximal portion 14 of the stiffener 12, such as second shoulder 91. Other suitable structures (not shown), for example a cooperating undercut and tab, etc., as known to the art, may also be used within the scope of this invention. According to one embodiment of the invention, the second magnetic part 79 is supported within the stiffener 12 such that relative rotational displacement between the second magnetic part 79 and the stiffener 12 is limited or prevented, e.g., by a keying arrangement (not shown). In another embodiment of the invention, at least while the first and second magnetic parts 78,79 are engaged, the proximal portion 14 of the stiffener 12 releasably engages the first magnetic part 78 such that relative rotational displacement between the first magnetic part 78 and the stiffener 12 is limited or prevented, e.g., with engaging structure as described above for first and second magnetic parts 78 and 79. In an embodiment of the invention (not shown), at least while the first and second magnetic parts 78,79 are engaged, the proximal portion 14 of the stiffener 12 releasably engages, directly or through a separate engagement device, the proximal portion of the tube-like structure such that relative rotational displacement between the tube-like structure and the stiffener 12 is limited or prevented. The second magnetic part 79 is sized for slidable displacement within at least the proximal portion 14 of the hollow stiffener 12, and is affixed to a retractable element or actuator, such as a wire or rod or filament 20. The distal end 22 of the wire or actuator extends beyond the distal end of the stiffener 12 to provide a portion manipulable to effect distal displacement of the wire or actuator 20, and with it, the second magnetic part 79 for disengaging the first and second magnetic parts.

In an embodiment of the invention, a mechanical device is included to provide a mechanical advantage to assist in separating the first and second magnetic parts 78, 79. In one embodiment, not shown, a lever is provided which includes a cam or cam-like surface. The lever is pivotally attached to a portion of the stiffener or other suitable structure and the distal portion of the actuator (wire, rod or filament) is fixed to the lever. Actuation of the lever provides axial displacement of the second magnetic part from a first position, in which the second magnetic part is engaged with the first magnetic part, to a second position in which the first and second magnetic parts are separated or disengaged.

The magnetic parts 78, 79 may comprise various magnetic materials and/or combinations thereof. For example, in one embodiment, magnetic parts 78 and 79 may both comprise permanent magnets. In another embodiment, one of the magnetic parts 78, 79 may comprise a permanent magnet and the other magnetizable material that is attracted by the permanent magnet. In another embodiment one or both parts 78, 79 may comprise magnetizable material that can be temporarily magnetized just prior to insertion. In another embodiment, an electromagnet is associated with the stiffener 12, and wires from the electromagnet extended therefrom through the internal passage 24 thereof and exit from the distal portion of the stiffener for connection to an electrical power source. Other magnetic systems will be evident to those of skill in the art from the disclosure herein.

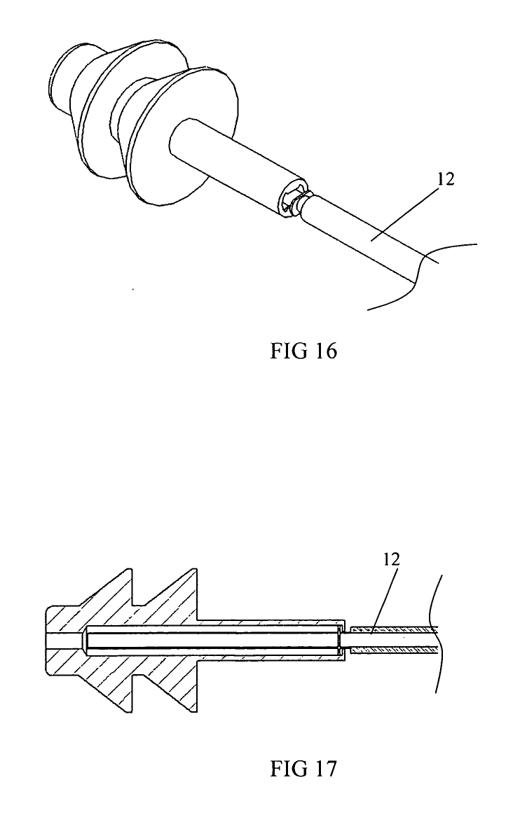

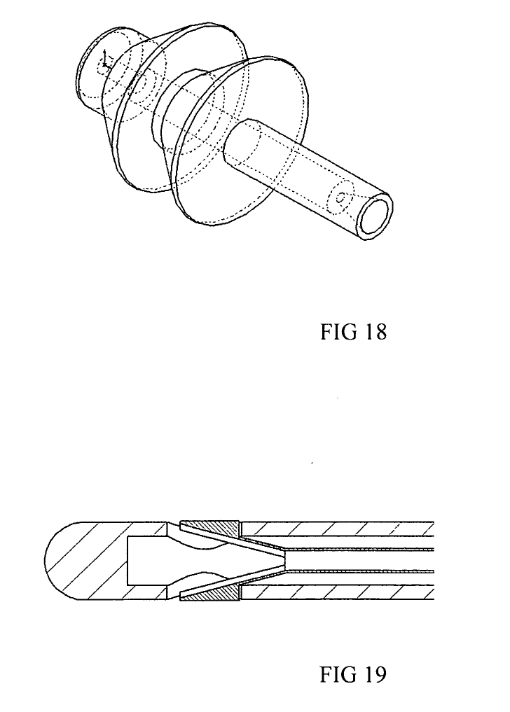

As illustrated in FIGS. 16 and 17, the proximal portion 14 of stiffener 12 may comprise a projection 92 comprising one or more lobes 93 adapted to engage a hole or recess 95 when the lobes 93 and recess 95 are in a certain rotational relationship. In an engaged position, the lobes 93 may rotate to functionally and releasable engage the stiffener 12 and the engagement device 18. In some embodiments, the lobes may rotate for a partial turn (less than)360° , preferably less than a half-turn)(180°), and further preferably approximately a quarter-turn)(90°). The recess 95 may include planar or helical surfaces configured to engage a planar surface or a similar helical surface on one or more of the lobes 93.

As illustrated in FIG. 18, the axial bore 34 of the engagement device 18 may include a tapered surface 31 adapted to releasably engage a tapered projection (not shown) of the stiffener. The functional engagement may resemble a tapered press fit similar to a luer connection.

As illustrated in FIG. 19, a proximal portion of a stiffener 12 according to an embodiment of the present invention may include one or more selectively expandable spring fingers 97 comprising spring finger tip 99 adapted to engage at least a portion of an opening in the distal portion of a tube-like structure, such as an eyelet 77 of a Foley catheter 70. An actuator, similar to the collar actuator described above, may be used to selectively radially collapse the spring fingers 97 for installation of the stiffener 12 within the tube-like structure. In the desired location, the actuator may radially expand the spring fingers 97, or allow a natural outward bias of the fingers 97 to radially expand the fingers, to engage the opening, or openings or eyelets 77 in the tube-like structure.

As disclosed herein, in some embodiments, the engagement device part of the facilitation device is provided as an insert to be inserted into a tube-like structure and in other embodiments the engagement device or engagement structure is formed integrally with the tube-like structure, generally in the proximal portion of the tube-like structure. As discussed above, the engagement device may comprise a locking wire system, a fastener system, a magnetic system, a mechanical system or connector (e.g., luer connectors, detents, press fits, screw threads, partial turn connectors, shot pins, buttons, cams, etc.) or other suitable releasable locking systems. The tube-like structure according to some embodiments include internal structure or structures to cooperate with associated structure or structures on the insertion facilitation device to provide releasable engagement with the insertion facilitation device. As discussed above, various releasable engagement arrangements, generally known in the art, can be employed to provide a suitable releasable engagement between the insertion facilitation device. The releasable engagement methods available with integrally formed engagement devices are similar to those available for use with a separately formed engagement device.

Embodiments of the disclosed invention have been described and illustrated in an exemplary and non-limiting sense, and are not to be limited to the precise details of methodology or construction set forth above. For example, variations and modifications of engagement devices, engaging arrangements, stiffeners, will be evident to those skilled in the relevant arts from the disclosure herein and are should be encompassed by the disclosure.

I claim:

1. An insertion facilitation device for facilitating insertion of a tube-like structure into a passage in a mammalian body, the device comprising:
(a) an elongate stiffener including a proximal end portion and a distal end portion, the stiffener being adapted to
extend in a lumen of the tube-like structure from a distal end portion thereof to a proximal end portion thereof, with the distal end portion of the stiffener at least partially within the distal end portion of the tube-like structure and the proximal end portion of the stiffener at least partially within the proximal end portion of the tube-like structure and
provide a stiffness to the tube-like structure that is greater with the stiffener than without the stiffener, and
be axially displaced within the tube-like structure relative to the tube-like structure; and
(b) an engagement device that is adapted in an engaging condition thereof to releasably engage the stiffener and tube-like structure with the stiffener extending within the lumen of the tube-like structure such that either or both axial and radial displacement of the stiffener relative to the tube-like structure is substantially eliminated during insertion of the tube-like structure into a body passage, and in a disengaging condition thereof to release engagement of the stiffener and tube-like structure such that the stiffener is at least axially displaceable within the lumen of the tube-like structure and can be withdrawn from the tube-like structure at least so that the proximal end portion of the stiffener is in or adjacent the distal end portion of the tube-like structure.

2. The insertion facilitation device according to claim 1 wherein the stiffener comprises a solid or hollow tube, rod, bar, wire, or an assembly of one or more solid or hollow tubes, rods, bars, or wires.

3. The insertion facilitation device according to claim 1 comprising a handle engaged with the distal portion of the stiffener.

4. The insertion facilitation device according to claim 3 wherein the handle is adapted to releasably engage the distal end portion of the tube-like structure.

5. The insertion facilitation device according to claim 1 wherein at least part of the engagement device is movable, the engagement device in a first condition thereof releasably engaging the stiffener and the tube-like structure and in a second condition thereof responsive to movement releasing engagement of the stiffener and the tube-like structure.

6. The insertion facilitation device according to claim 1 wherein the engagement device is adapted to be inserted into a tube-like structure and engaged with the proximal end portion thereof.

7. The insertion facilitation device according to claim I wherein the engagement device comprises:
a first outer diameter portion configured to engage a portion of the lumen at the proximal portion of the tube-like structure and at least substantially prevent at least one of radial motion and axial motion in a distal direction with respect to the lumen; and
a portion of the engagement device engaging at least a portion of the proximal end portion of the stiffener.

8. The insertion facilitation device according to claim 7 wherein the engagement device comprises:
a second outer diameter portion smaller that the first outer diameter portion;
an inner bore at least in the second outer diameter portion;
at least one radial opening between the inner bore and the second outer diameter portion; and
wherein the stiffener further comprises:
an outer surface;
an axial bore extending from the distal end portion toward the proximal end portion;
at least one radial opening between the inner bore and the outer surface configured to substantially align with the at least one radial opening in the engagement device; and wherein the insertion facilitation device further comprises:
a locking wire adapted for axial movement within the axial bore of the stiffener from the distal end portion to the at least one pair of aligned openings, the locking wire extending at least partially through the aligned openings and releasably engaging the engagement device and the stiffener.

9. The insertion facilitation device according to claim 7 wherein the distal end portion of the locking wire includes a handle.

10. The insertion facilitation device according to claim 5 wherein the engagement device is engaged with the proximal end portion of the stiffener and is adapted to releasably engage the lumen of the tube-like structure in the proximal portion thereof.

11. The insertion facilitation device according to claim 5 wherein the engagement device is engaged with the proximal end portion of the stiffener and is adapted to releasably engage a structural feature of the tube-like structure.

12. The insertion facilitation device according to claim 11 wherein the engagement device is engaged with at least one opening to the lumen of the tube-like structure at the proximal end portion thereof.

13. The insertion facilitation device according to claim 11 wherein the engagement device comprises a selectively expandable resilient spring arm.

14. The insertion facilitation device according to claim 11 wherein the engagement device comprises a selectively expandable malecot.

15. The insertion facilitation device according to claim 1 wherein the tube-like structure comprises at least a first element of the engagement device and the stiffener comprises at least a second element of the engagement device adapted to releasably engage the first element.

16. The insertion facilitation device according to claim 15 wherein the first element of the engagement device comprises a first magnetic material having a first interface surface and the second element of the engagement device comprises a second magnetic material having a second interface surface and having appropriate polarity to create a magnetic attraction with the first magnetic material sufficiently strong to engage the first and second interface surfaces.

17. The insertion facilitation device according to claim 16 wherein the first interface surface includes features configured to cooperate with features included on the second interface surface such that the cooperating features resist relative angular movement between the first and second magnetic materials.

18. The insertion facilitation device according to claim 16 wherein the second element of the engagement device is a second magnetic material having a second interface surface and having appropriate polarity to create a magnetic attraction with the first magnetic material wherein the second magnetic material is slidably disposed with respect to a proximal portion of the stiffener such that the second magnetic material may be selectively advanced proximally to engage with the first magnetic material or retracted to disengage from the first magnetic material.

19. The insertion facilitation device according to claim 18 wherein the selective advancement and retraction of the second magnetic material is effected by an actuator configured for axial displacement within the axial bore of the lumen.

20. The insertion facilitation device according to claim 15 wherein the first element of the engagement device comprises a first recess or a first projection and the second element comprises a second projection or a second recess suitable to engage the first element.

21. The insertion facilitation device according to claim 20 wherein a projection comprises at least a partial helical thread form and a recess comprise at least a mating partial helical thread form.

22. The insertion facilitation device according to claim 15 wherein the first element of the engagement device comprises a first half of a luer fitting and the second element of the engagement device comprises a second half of a luer fitting.

23. A method for providing stiffness to a tube-like structure during insertion into a passage, the method comprising:
introducing a stiffener to a lumen of a tube-like structure through an opening at the distal end of the tube-like structure;
advancing the stiffener axially towards the proximal portion of the tube-like structure; and
actuating an engagement device releasably engaging directly or indirectly the stiffener to a selected portion of the lumen in the tube-like structure.

24. A catheter system for facilitating insertion of a catheter into a body passage of a mammalian body, the system comprising:
a catheter:
an elongate stiffener including a proximal end portion and a distal end portion, the stiffener extendable into the catheter from a distal end portion thereof to a proximal end portion thereof, with the distal end portion of the stiffener at least partially within the distal end portion of the catheter and the proximal end portion of the stiffener at least partially within the proximal end portion of the catheter and being axially displaceable within the catheter relative to the catheter; and
an engagement device releasably engaging the stiffener and the catheter with the stiffener extending within the catheter such that when the stiffener and catheter are engaged either or both axial and radial displacement of the stiffener relative to the catheter is substantially eliminated, and when the stiffener and catheter are not engaged, the stiffener is at least axially displaceable within the catheter and can be withdrawn from the catheter at least to the extent that the proximal end portion of the stiffener is in or adjacent the distal end portion of the catheter, wherein at least part of the engagement device is movable, the engagement device in a first condition thereof releasably engaging the stiffener and the catheter and in a second condition thereof responsive to movement releasing engagement of the stiffener and the catheter.

25. The catheter system of claim 24 wherein at least part of the engagement device engages a portion of the catheter.

26. The catheter system of claim 24 wherein at least part of the engagement device releasably engages a portion of the catheter.

27. The catheter system of claim 24 wherein at least part of the engagement device is releasably engaged to the proximal end portion of the stiffener and is adapted to be axially displaced within the catheter relative to the catheter from a distal portion of the catheter to a proximal portion of the catheter and engage a proximal portion of the catheter.
